(19) United States
(12) Patent Application Publication
(10) Pub. No.: US 2005/0137206 A1
(43) Pub. Date: Jun. 23, 2005

(54) Method for treatment of anxiety and depression

(76) Inventors: Joseph P. Yevich, Southington, CT (US); Robert F. Mayol, Burns, TN (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/969,511

(22) Filed: Oct. 20, 2004

Related U.S. Application Data
(63) Continuation-in-part of application No. 10/201,733, filed on Jul. 23, 2002.
Application No. 10/201,733 is a continuation-in-part of application No. 09/588,220, filed on Jun. 6, 2000, now abandoned.
Application No. 09/588,220 is a continuation-in-part of application No. 09/484,161, filed on Jan. 18, 2000, now abandoned.
Application No. 09/484,161 is a division of application No. 09/368,842, filed on Aug. 5, 1999, now abandoned.

(51) Int. Cl. A61K 31/506

(52) U.S. Cl. 514/252.15

(57) ABSTRACT

An improved method of treating anxious and/or depressed patients involves direct administration of pharmaceutically purified 6-hydroxy-8-[4-[4-(2-pyrimidinyl)-piperazinyl]-butyl]-8-azaspiro[4.5]-7,9-dione(6-hydroxybuspirone) or a pharmaceutically acceptable salt or hydrate thereof formulated in appropriate pharmaceutical compositions to patients in need of such treatment.

### CROSS REFERENCE TO RELATED APPLICATIONS

This application is a continuation-in-part of pending application Ser. No. 10/201,733 filed Jul. 23, 2002, which is a continuation-in-part of the now abandoned application Ser. No. 09/588,220 filed Jun. 6, 2000, which is an abandoned continuation-in-part of application Ser. No. 09/484,161 filed Jan. 18, 2000, which is an abandoned divisional application of application Ser. No. 09/368,842 filed Aug. 5, 1999, now abandoned.

### BACKGROUND OF THE INVENTION

This disclosure and claims concern an improved process for the alleviation of anxiety and/or depression by treatment with pharmaceutical compositions comprising therapeutically effective amounts of pharmaceutically purified 6-hydroxy-8-[4-[4-(2-pyrimidinyl)-piperazinyl]-butyl]-8-azaspiro[4.5]-7,9-dione. This compound itself was first disclosed by Jajoo, et al., Drug Metab. and Disposition, 17/6, pp. 634-640, 1989, as being one of several metabolites of the clinically useful drug, buspirone. Confirmation of molecular structure for this urinary metabolite was achieved by comparison with an authentic sample of the compound prepared synthetically. This metabolite compound is also known as BMY 28674, BMS 528215, and as 6-hydroxybuspirone (6-HB).

Buspirone, chemically: 8-[4-[4-(2-pyrimidinyl)1-piperazinyl]butyl-8-azaspiro(4,5)-decane-7,9-dione, is a pharmaceutically active compound found to be effective for the treatment of anxiety disorders and depression. It has been generally accepted that buspirone exerts its pharmacologic effects through the serotonin 1A (5-HT1A) receptor. However, buspirone shows a very high first pass metabolism and, in general, only about 4% of a therapeutic dose of buspirone reaches the systemic circulation in non-metabolized form after oral administration (Mayol, et al., Clin. Pharmacol. Ther., 37, p. 210, 1985). Large differences in buspirone absorption both between doses and between individuals have also been observed. This has been demonstrated by variations of the maximum plasma concentration of drug in individuals by up to 10-fold differences (Gammans, et al., American J. Med., 80, Suppl. 3B, pp. 41-51, 1986).

The synthesis of buspirone and related analogs and disclosure of their psychotropic properties were first described by Wu, et al., in U.S. Pat. No. 3,717,634. The use of buspirone hydrochloride as a novel antianxiety agent for the treatment of neurotic patients, with and without accompanying depression, has been described by Casten, et al., in U.S. Pat. No. 4,182,763. Subsequent clinical use established buspirone as an effective agent for treating symptoms of anxiety and/or depression.

Prior to the present work there was no knowledge of the blood level concentrations or possible therapeutic effects of 6-hydroxybuspirone (6-HB). In fact, no significant biological activity of any type has ever been disclosed for 6-HB which had been regarded as an excretory metabolite found chiefly in urine. With the exception of the metabolite 1-pyrimidinylpiperazine (1-PP), no significant pharmacologic activity has been disclosed for any of the known buspirone metabolites. See: VanderMaalen, et al., Eur. J. Pharmacol., 129, pp. 123-130, 1986; Gammans, et al., JAMA, (March, 1986), Vol. 80, Supp. 3B, pp. 43-44. Two buspirone metabolites: 1-PP and a different monohydroxyl buspirone (pyrimidinyl ring hydroxylation), had been found in significant amounts in the blood of humans given buspirone. This isomeric monohydroxylated metabolite (a position isomer of 6-HB, denoted 5-hydroxy-pyrimidinyl buspirone metabolite) was believed to be the metabolite most likely to effect a pharmacologic response by virtue of its presence in much higher concentrations in the blood stream than buspirone and by its ability to immunoprecipitate with buspirone. Assuming that the 5-HT tissue receptor would bear some similarity to the immunoassay's antibody binding site strongly suggested that the 5-hydroxy-pyrimidinyl-metabolite would be the most promising metabolite as far as being biologically similar to buspirone. Pharmacologic screening, however, failed to show any useful pharmacologic activity for this metabolite. As a consequence, oral dosing of buspirone for treating anxiety and depression has been believed to be optimized when done in a manner to maximize the concentration of unchanged drug at the expense of metabolites. In effect, previous references acted to teach away from making and using the present 6-HB pharmaceutical compositions. The prior art, taken as a whole, would discourage one skilled in the art from pharmacologic use of buspirone metabolites such as 6-HB.

In U.S. Pat. No. 5,431,922 an extended-release formulation of buspirone was described as providing an improvement in oral administration of the drug on the basis that blood levels of unchanged buspirone were increased while metabolite levels were reduced as measured by the ratio of plasma levels of buspirone to the 1-PP metabolite. However, no efficacy data were ever disclosed for these ER formulations nor were they ever commercialized.

U.S. Pat. No. 5,633,009 disclosed and claimed a transdermal patch for delivering buspirone. The transdermal delivery, as expected, reliably gave higher buspirone blood levels (AUC) with much reduced metabolite levels, as measured by 1-PP. A typical patch was designed to deliver 60 mg of buspirone per 24 hour period. Surprisingly, clinical studies in anxious patients, with and without accompanying depression, did not result in any clinical benefit from the buspirone patch that was distinguishable from placebo.

Most recently an improved method for oral administration of buspirone was claimed in U.S. Pat. No. 6,008,222 wherein the bioavailability of unchanged buspirone is increased and metabolite formation is decreased. The disclosed method involved co-administration of buspirone with the drug nefazodone, an inhibitor of cytochrome P4503A4 (CYP3A4). Based on assessment of preliminary clinical data, no further development of this drug combination pharmaceutical formulation has been planned.

In summary, prior art teachings have subscribed to the widely accepted principle that buspirone's drug effect was maximized by suppression of buspirone's metabolism. While the urine-derived metabolite, 6-HB, had been known to be one of several human metabolites resulting from oral administration of buspirone, no useful pharmacologic activity has previously been associated with the compound itself prior to the present work. And further, no useful biologic activity was expected for 6-HB since another isomeric monohydroxylated metabolite, which was found in plasma and which immunoprecipitated with buspirone and was therefore considered to be the most likely of all the metabolites to possess buspirone-like properties, failed to show any pharmacologic activity in animal tests. As a consequence, no pharmaceutical compositions or dosage forms for clinical use comprising 6-HB were ever developed.

The unexpected discovery of activity for 6-HB in animal models of anxiety, along with the results of determinations of 6-HB blood levels in humans, has led to the conclusion that 6-HB is the active metabolite of buspirone. As a result, there has been development of pharmaceutical compositions and dosage forms containing pharmaceutically purified 6-HB which are intended for administration to patients suffering from anxiety and/or depression. These pharmaceutical compositions should provide a more robust and reliable treatment for those patients due to the delivery of effective amounts of 6-HB compared to the varying amounts previously delivered via buspirone metabolism.

### BRIEF DESCRIPTION OF THE DRAWINGS

FIG. 1. Effect of 6-HB on isolation-induced ultrasonic vocalization and locomotor activity in the rat pup.

FIG. 2. Effect of buspirone on isolation-induced ultrasonic vocalization and locomotor activity in the rat pup.

FIG. 3. Human blood level concentrations of buspirone following oral dosing of buspirone in human subjects.

FIG. 4. Human blood level concentrations of 1-PP following oral dosing of buspirone in human subjects.

FIG. 5. Human blood level concentrations of 6-HB following oral dosing of buspirone in human subjects.

### SUMMARY AND DETAILED DESCRIPTION OF THE INVENTION

We have discovered that 6-hydroxy-8-[4-[4-(2-pyrimidinyl)piperazinyl]-butyl]-8-azaspiro[4.5]-7,9-dione (I) is the active metabolite of buspirone and is referred to herein as 6-hydroxybuspirone or 6-HB. The compound has the following structural formula: 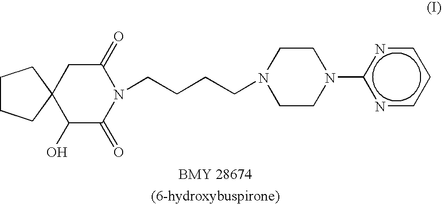
and as the active metabolite of buspirone, would be more useful for the treatment of anxiety and/or depression than buspirone itself. The advantage of 6-hydroxybuspirone administration is that higher and more reliable blood levels can be achieved than by administration of buspirone itself, due to variability between patients of buspirone's metabolism to 6-HB, the active agent. By direct administration of pharmaceutically purified 6-HB, a more reliable and robust psychopharmacologic clinical response can be elicited in patients suffering from anxiety and/or depression. By anxiety and/or depression is meant anxiety, anxiety mixed with depression, depression, and depression mixed with anxiety.

The results of previous testing of the clinically useful anxiolytic agent buspirone, and several of its putative and actual metabolites, led to a conclusion that the pharmacologic action was mainly provided by buspirone itself with little, if any, contribution being made by the various buspirone metabolites. For example, systemic administration (I.V. and intragastric) of certain putative metabolites to rats resulted in little to no inhibition of dorsal raphe neuronal firing. In contrast, buspirone itself potently inhibits the firing of dorsal raphe neurons. See: VanderMaelen, et al., Eur. J. Pharmacol., 129, pp. 123-30, 1986. While one metabolite, the structural fragment 1-(2-pyrimidinyl)piperazine, also known as 1-PP, 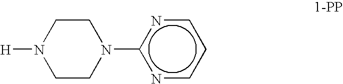
did show weak inhibition of dorsal raphe firing as well as eliciting some antianxiety activity in certain other preclinical tests (see: U.S. Pat. No. 4,409,223), it also displayed anxiogenic properties in other behavioral testing paradigms. See: Cervo, et al., Life Sciences, 43, pp. 2095-2102, 1988; Martin, Psychopharmacology, 104, pp. 275-278, 1991. It is probable that the biological effect of 1-PP is mediated through an alpha 2 adrenergic mechanism as 1-PP does not demonstrate binding at the 5-HT1A receptor. Presently, the clinical effect of 1-PP is unclear and no use of 1-PP for treating anxiety and/or depression has been made.

With general acceptance that the active pharmacologic agent is buspirone itself, drug administration was done in a manner for maximizing blood levels of unchanged buspirone. In patients where metabolism of buspirone is inhibited, either because of the patient's hepatic enzymatic activity levels or because of ingestion of substances that have inhibitory effects on hepatic metabolism, particularly CYP3A4; patients were advised to reduce the amount of buspirone being taken for fear of adverse effects with higher buspirone blood levels. This, of course, had the effect of minimizing blood levels of the active metabolite 6-HB. With discovery of 6-HB as the pharmacologically active metabolite, metabolically compromised patients can be more effectively treated by direct administration of pharmaceutically purified 6-HB instead of buspirone.

While regarded as an effective treatment of anxiety and depression, buspirone does not provide relief for a certain percentage of patients. This lack of efficacy has previously been ascribed to insufficient blood levels of unchanged buspirone being achieved in non-responders even though buspirone blood levels are low in all patients. Scientific confirmation of this hypothesized explanation for buspirone treatment failure is lacking. An alternate explanation emerges in light of the discovery of the active metabolite 6-HB and its pharmacologic effect. A more likely explanation for treatment failure or a minimal response from buspirone in certain patients can be ascribed to low blood levels of the pharmacologically active 6-HB. Non-responders given buspirone can be seen as patients whose metabolic conversion of buspirone is insufficient to achieve efficacious levels of 6-HB. Support for this explanation can be found with the observation of wide variability of buspirone metabolism as observed with buspirone blood levels seen both within the same patient and between patients following oral buspirone administration. This metabolic variability can be due to genetic variation in individuals as well as from variations that occur in activity levels of human hepatic metabolism during the course of daily living.

There has also been a time lag observed for onset of therapeutic effect following initiation of buspirone treatment. This delay could be due to the time required for 6-HB accumulation as well as re-regulation of receptor site dynamics. The dependence of clinical effect on the appearance of sustained levels of the metabolite 6-HB and its subsequent receptor dynamics could better explain the clinical observations of a time delay seen following oral administration of buspirone to patients.

On the basis of the accepted rationale that intact buspirone provided the useful pychopharmacologic activity seen clinically, a transdermal patch delivery system for buspirone was developed (see: U.S. Pat. No. 5,633,009). A buspirone transdermal patch was predicted to be a superior treatment for anxiety and depression since transdermal drug delivery minimized metabolism of buspirone, thereby giving significantly larger amounts of parent drug with much reduced levels of its metabolites. Surprisingly, little to no clinical effect was seen with patient treatment by the buspirone transdermal patch. This unexpected result has led to reevaluation of buspirone metabolites and ultimately to the discovery of 6-HB's high blood levels in humans and of its pharmacologic activity.

The following metabolic scheme (Scheme 1) for buspirone is taken from Jajoo, et al., Xenobiotica, 1990, Vol. 20, No. 8, pp. 779-786, “In vitro metabolism of the antianxiety drug buspirone as a predictor of its metabolism in vivo.”

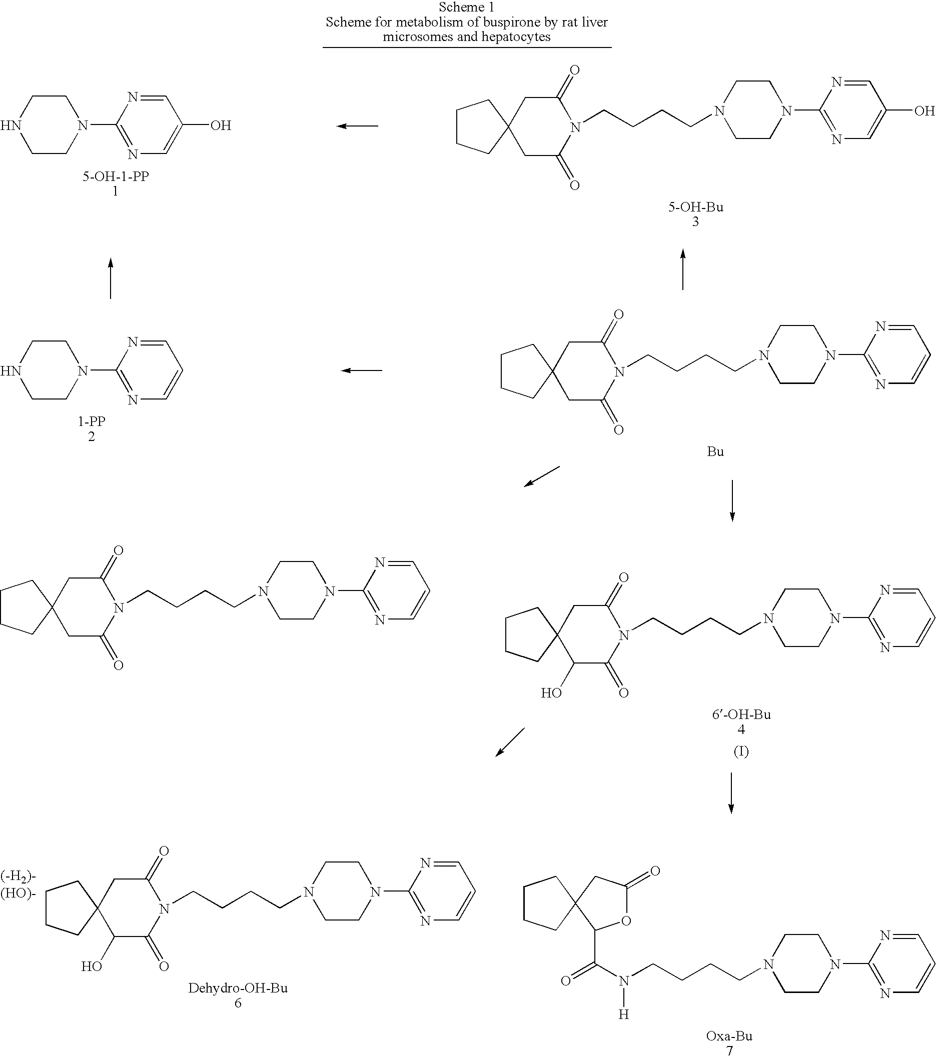

The work culminating in discovery of the pharmacologic activity of 6-HB began by evaluation of relevant receptor binding of buspirone metabolites. Accordingly, the in vitro activity of buspirone (Bu; MJ 9022) and its metabolites 1-PP (BMY 13653), 3′-OH-buspirone (BMY 14295), 5-OH-buspirone (BMY 14131), and 6′-OH-buspirone (6-HB; BMY 28674); were evaluated for activity at the human 5-HT1A receptor. Results of these experiments are found in Table 1.

TABLE 1

| Compound | IC₅₀ [nM] | STDEV | Kᵢ value | N |
|---|---|---|---|---|
| 8-OH-DPAT (reference) | 2.5 | 0.9 | 1 | 8 |
| Buspirone (MJ 9022) | 30 | 18 | 15 | 8 |
| 6-OH-buspirone (BMY 28674) | 114 | 85 | 57 | 7 |
| 5-OH-buspirone (BMY 14131) | 928 | 176 | 464 | 7 |
| 3-OH-buspirone (BMY 14295) | 652 | 402 | 326 | 7 |
| 1-PP (BMY 13653) | >1000 | — | — | 3 |

As can be seen, Table 1 summarizes the in vitro action of MJ 9022 (buspirone) and its metabolites BMY 13653 (1-PP), BMY 14131 (5-OH-buspirone), BMY 14295 (3-OH-buspirone), and BMY 28674 (6-OH-buspirone) at the human serotonin 1A (5-HT1A) receptor. Buspirone demonstrates a high affinity for the human 5-HT1A receptor (Kᵢ=15 nM). 6-HB (BMY 28674) has a binding affinity that approaches that of buspirone (Kᵢ=57 nM). The other metabolites tested have relatively weak affinity for human 5-HT1A receptor compared to buspirone.

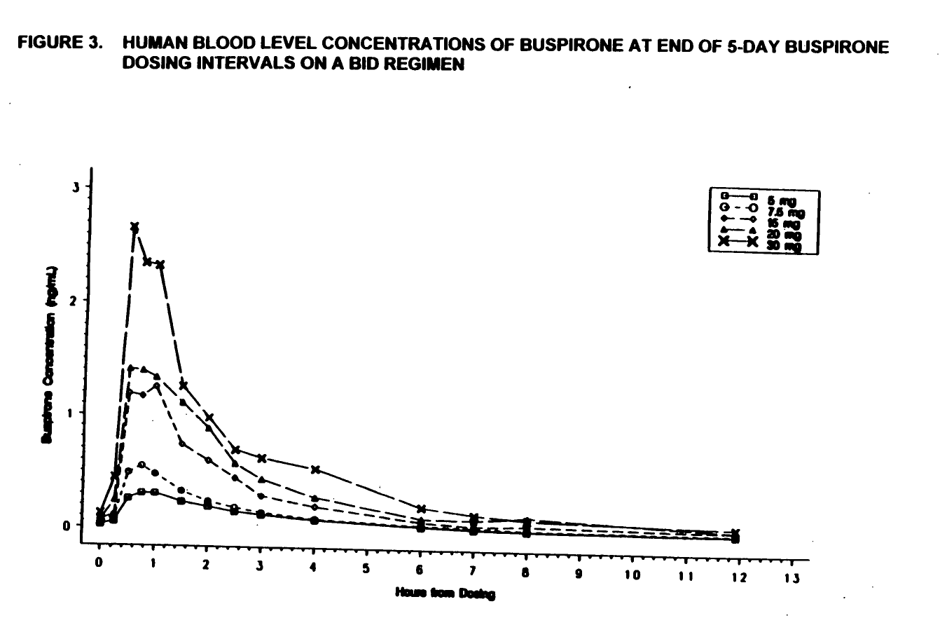

6-HB appears to be the active metabolite of buspirone. Not only is it the second most abundant metabolite following buspirone administration to be found in human urine (5-hydroxy-1 PP being most 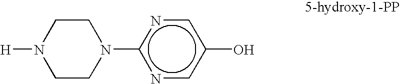
abundant); but more importantly, human blood levels of 6-HB are about 40 times greater than those of buspirone and several-fold greater than those of 1-PP (see FIGS. 3-5). Also of significance is the fact that the skeletal structure of buspirone remains intact in 6-HB. In addition, binding data at the 5-HT1A receptor indicates that 6-HB has a binding affinity closer to that of buspirone in contrast to the other buspirone metabolites which demonstrate only weaker interaction at the 5-HT1A site. The 5-HT1A receptor is a serotonergic receptor and regulation of the serotonergic system is intimately involved with anxiety and depression. The active metabolite research has focused on those metabolites that maintain the buspirone skeletal structure and that have no more than one hydrophilic hydroxy group incorporated into the molecule. The presence of more than one hydrophilic hydroxy group is likely to reduce the distribution and transport of those poly-hydroxylated metabolic products into the CNS regions of the body thereby making requisite receptor interactions unlikely to occur in target regions. For in vivo proof of principle, a sensitive anxiolytic animal paradigm was selected for testing 6-HB for serotonergic activity.

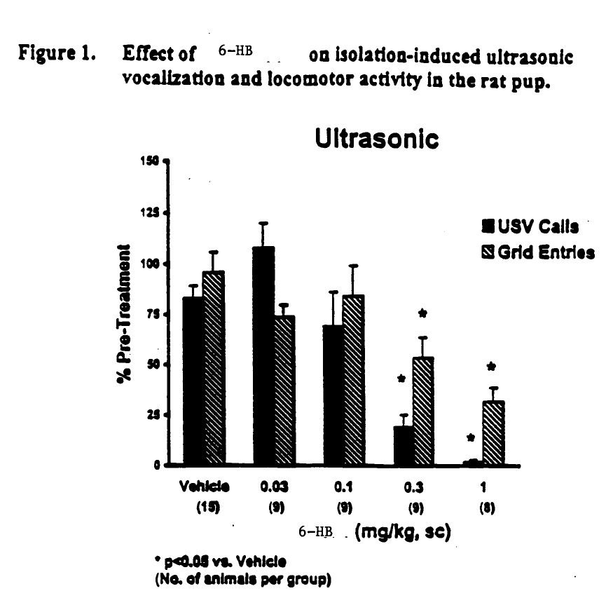

Ultrasonic vocalizations emitted by rat pups following isolation from their mother and littermates and subjected to a variety of environmental stimuli (e.g., cold temperature) has proven to be a sensitive method for assessing potential anxiolytic and anxiogenic compounds (Winslow and Insel, 1991, Psychopharmacology, 105:513-520). Psychoactive compounds purported to have anxiolytic activity suppress the frequency of ultrasonic calls whereas calls are increased by drugs with anxiogenic properties. More importantly, the isolation-induced ultrasonic vocalization paradigm appears to be most sensitive for detecting anxiolytic properties across a broad spectrum of drug classes such as benzodiazepines, 5-HT reuptake inhibitors, 5-HT1A agonists as well as NMDA antagonists. In the present investigation, the 6-hydroxylated metabolite of buspirone, 6-HB, which has affinity for the human 5-HT1A receptor (Kᵢ=57 nM) was assessed for potential anxiolytic activity using 9-11 day old rat pups that had been separated from their mother and littermates and placed on a cold (18-20° C.) plate to elicit distress-induced ultrasonic vocalizations. See FIG. 1.

Administration of 6-HB (0.03-1 mg/kg, sc; FIG. 1) 30 min prior to test produced a dose-dependent suppression of rat pup ultrasonic vocalization on the cold plate [F(4,45)=19.27, p=0.0001]. The dose of 6-HB predicted to reduce the number of calls by 50% (ID₅₀) was 0.13 mg/kg. Locomotor activity was also significantly impaired following 6-HB [F(4,45)=5.85, p=0.007]. However, the ID₅₀ dose (0.41 mg/kg) of 6-HB estimated to reduce locomotor activity was approximately 3-fold greater than the ID₅₀ dose (0.13 mg/kg) observed for suppressing ultrasonic calls suggesting that the anxiolytic properties of 6-HB occur at lower doses than those reducing locomotor activity.

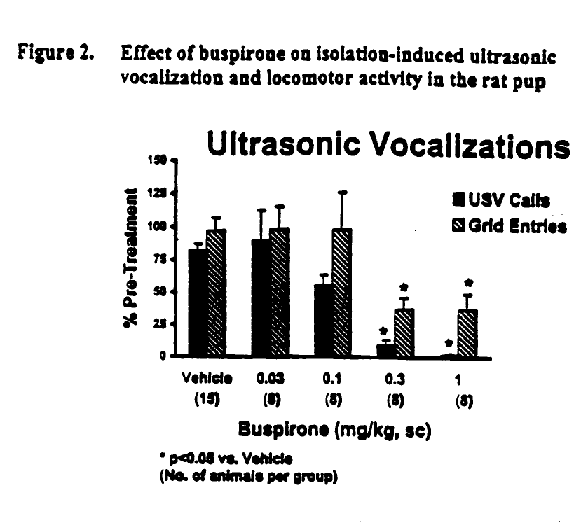

Administration of buspirone (0.03-1 mg/kg, sc; FIG. 2) 30 min prior to test produced a dose-dependent suppression of rat pup ultrasonic vocalization on the cold plate [F(4,42)=15.44, p=0.0001]. The dose of buspirone predicted to reduce the number of calls by 50% (ID₅₀) was 0.10 mg/kg. Locomotor activity was also impaired [F(4,42)=4.343, p=0.005] at approximately 5-fold greater doses than those suppressing ultrasonic calls.

The present in vivo proof of principle test results demonstrate that, like buspirone, the metabolite 6-HB elicits anxiolytic-like activity in the rat pup isolation-induced ultrasonic vocalization model of anxiety. The anxiolytic activity associated with 6-HB (and buspirone) occurred at much lower doses than those required to suppress motor activity. In summary, the foregoing in vitro and in vivo tests demonstrate positive antianxiety test results for both buspirone and 6-HB; however, buspirone blood level concentrations are minimal following oral administration of buspirone to human subjects. Prior to the present work, no information regarding clinical blood level concentrations of 6-HB existed.

Human pharmacokinetic studies further support the role of 6-HB as the pharmacologically active metabolite of buspirone.

Human subjects (n=13) were administered buspirone orally for 25 days with total daily doses ranging from 10 mg to 60 mg. The dosing schedule was divided into five 5-day dose intervals with BID dosing being increased in each interval. Pharmacokinetic measurements were made on day 5 of each interval and these data were used to assess the pharmacokinetics of buspirone, 1-PP, and 6-HB. The human dosing schedule is shown below.

| Dosing | Buspirone | PK Measurement |
|---|---|---|
| Interval | BID Dose (mg) | (Study Day) |
| 1 | 5 | 5 |
| 2 | 7.5 | 10 |
| 3 | 15 | 15 |
| 4 | 20 | 20 |
| 5 | 30 | 25 |

These multiple doses of oral buspirone at the five dose levels were found to be safe and generally well tolerated in the healthy adults participating in the 25-day study.

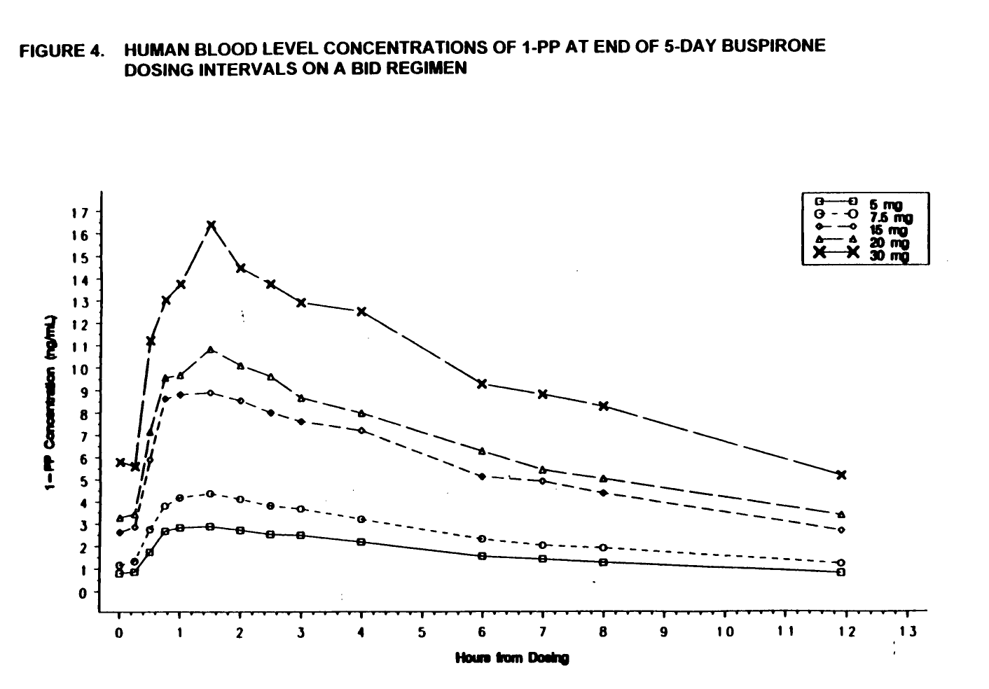

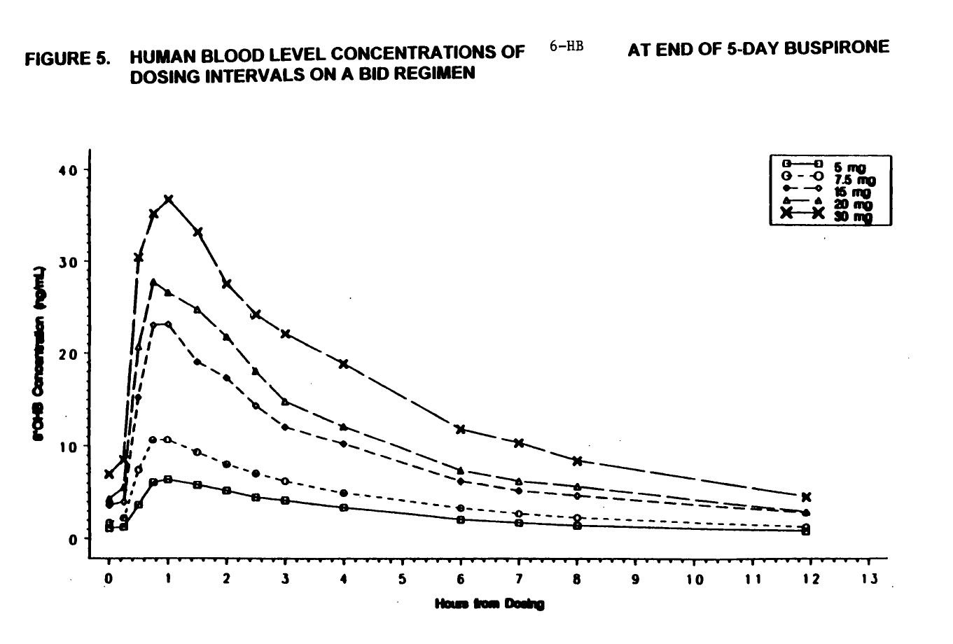

FIGS. 3, 4 and 5 show mean blood level concentrations of buspirone, 1-PP, and 6-HB, respectively, over a 12-hour dosing period on the last day of each dosing interval. Buspirone levels (FIG. 3) are in general very low (about 1-2 ng/mL at the higher doses) and drop to less than 1 ng/mL levels two hours post-dose. In contrast, 1-PP levels (FIG. 4) and 6-HB levels (FIG. 5) are much higher and are sustained compared to buspirone. 6-HB has several-fold higher concentrations than 1-PP and about 30 to 40-fold higher concentrations than buspirone.

Such studies indicate that after oral administration of buspirone, it is blood levels of the metabolite, 6-HB, that are more meaningful compared to the negligible blood levels observed for buspirone. Although buspirone itself has been demonstrated to have psychopharmacologic properties in test models such as the rat pup USV model described herein, the low blood level concentrations seen in humans leads to the conclusion that it is the abundant metabolite 6-HB that mediates the pharmacologic effects seen clinically. Prior to this present evaluation of buspirone metabolites, the relative abundance of 6-HB in humans following oral-administration of buspirone was not known.

Therefore, it can be seen that the administration of pharmaceutically purified 6-HB represents an improved method of treating anxiety and/or depression, compared to administration of buspirone. This improvement results from providing effective blood levels of 6-HB in anxious and/or depressed patients. The optimum means to achieve this would be by the administration of pharmaceutically purified 6-HB itself to the patient. Therefore, one aspect of the present discovery concerns the process for ameliorating anxiety and/or depression in a human in need of such treatment by administration of a clinically effective dose of pharmaceutically purified 6-HB.

An effective dose should, in general, provide minimum blood level concentrations (CMIN) of 6-HB that are at least 1 to 2 ng/mL. Generally the point of measurement for CMIN levels is 12 hours post-dose; i.e., just before the next BID dose. 6-HB can be administered by a variety of routes including, but not limited to, oral; sublingual; buccal; transnasal; or parenteral, e.g. intramuscular, intravenous, subcutaneous, etc. Therapeutically, 6-HB can be given by one of these routes as a formulation comprised of an effective amount of 6-HB, or one of its pharmaceutically acceptable acid addition salts or a hydrate, in a pharmaceutically acceptable carrier. Another aspect of the present discovery concerns pharmaceutical formulations and dosage forms for convenient and effective administration of 6-HB. Pharmaceutical compositions which provide from about 5 to 50 mg of the active ingredient per unit dose are preferred and can be conventionally prepared as aqueous solutions and aqueous or oily suspensions. 6-HB can also be given orally when compounded in an oral dosing formulation such as a tablet, lozenge, capsule, syrup, elixir, aqueous solution or suspension. Oral administration of 6-HB is preferred.

The pharmaceutically acceptable acid addition salts of 6-HB are also considered useful for use in pharmaceutical formulations. By definition, these are those salts in which the anion does not contribute significantly to toxicity or pharmacological activity of the base form of 6-HB.

Acid addition salts are obtained either by reaction of 6-HB with an organic or inorganic acid, preferably by contact in solution, or by any of the standard methods detailed in the literature and available to any practitioner skilled in the art. Examples of useful organic acids are carboxylic acids such as maleic acid, acetic acid, tartaric acid, propionic acid, fumaric acid, isethionic acid, succinic acid, pamoic acid, and the like; useful inorganic acids are hydrohalide acids such as HCl, HBr, Hl; sulfuric acid; phosphoric acid; and the like.

A second aspect of the present discovery deals with the 6-HB pharmaceutical compositions themselves. The preferred oral compositions are in the form of tablets or capsules and in addition to 6-HB may contain conventional excipients such as binding agents (e.g., syrup, acacia, gelatin, sorbitol, tragecanth, or polyvinyl pyrrolidone), fillers (e.g., lactose, sugar, maize-starch, calcium phosphate, sorbitol or glycine), lubricants (e.g., magnesium stearate, talc, polyethyleneglycol or silica), disintegrants (e.g., starch), and wetting agents (e.g., sodium lauryl sulfate). Solutions or suspensions of 6-HB with conventional pharmaceutical vehicles are employed for parenteral compositions such as an aqueous solution for intravenous injection or an oily suspension for intramuscular injection. Such compositions having the desired clarity, stability and adaptability for parenteral use are obtained by dissolving from 0.1 % to 10% by weight of the active ingredient (6-HB or a pharmaceutically acceptable acid addition salt or hydrate thereof) in water or a vehicle consisting of a polyhydric aliphatic alcohol such as glycerine, propylene glycol, and polyethylene glycols or mixtures thereof. The polyethylene glycols consist of a mixture of non-volatile, normally liquid, polyethylene glycols which are soluble in both water and organic liquids and which have molecular weights from about 200 to 1500. 6-HB can be synthesized conveniently from buspirone and buspirone may be synthesized by methods readily available in the chemical literature and known to one skilled in synthetic organic chemistry. One method of preparation utilizing buspirone as a starting material is shown in Scheme 2. An improved single-pot process starting with buspirone is shown in Scheme 3.

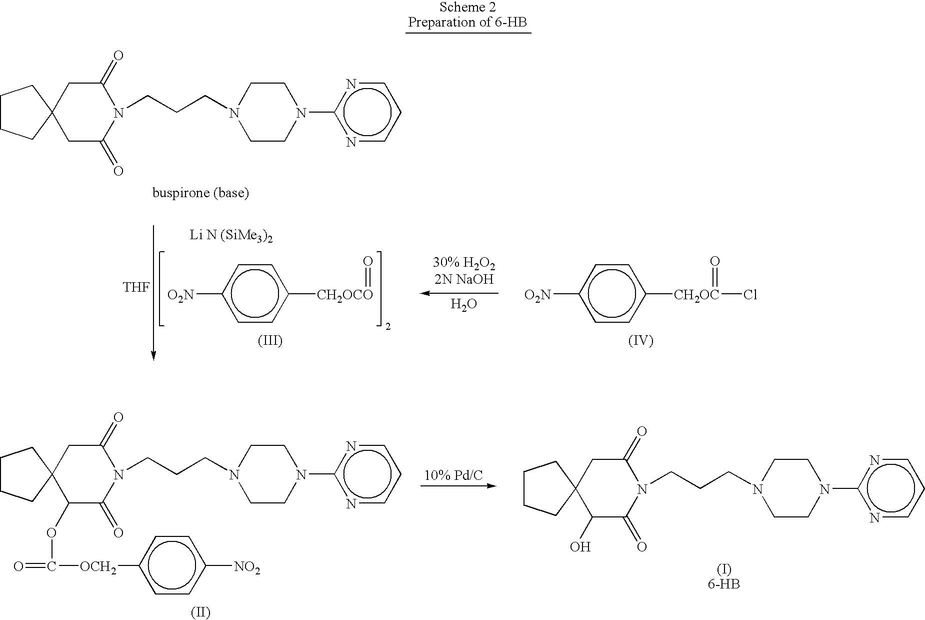

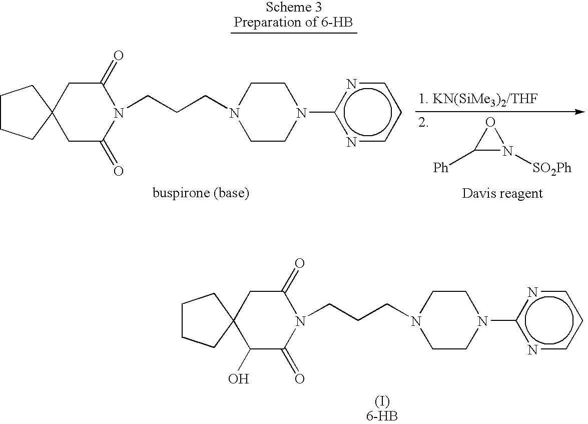

### DESCRIPTION OF SPECIFIC EMBODIMENTS

The compound whose use constitutes this invention and its method of preparation will appear more fully in light of the following examples which are given for the purpose of illustration only and are not to be construed as limiting the invention in sphere or scope.

### Example 1

### Preparation of 6-Hydroxybuspirone (I)

A. Di4-nitrobenzyl peroxydicarbonate (III)

Di-4-nitrobenzyl peroxydicarbonate was prepared using a modification of the literature procedure¹. Thus, to an ice-cold solution of 4-nitrobenzyl chloroformate (10.11 g, 4.7 mmol) in acetone (20 mL) was added dropwise over 30 min an ice-cold mixture of 30% H₂O₂ (2.7 mL, 24 mmol) and 2.35 N NaOH (20 mL, 47 mmol). The mixture was vigorously stirred for 15 min and then it was filtered and the filter-cake was washed with water and then with hexane. The resulting damp solid was taken up in dichloromethane, the solution was dried (Na₂SO₄) and then it was diluted with an equal volume of hexane. Concentration of this solution at 20° C. on a rotary evaporator gave a crystalline precipitate which was filtered, washed with hexane and dried in vacuo to give compound III (6.82 g, 74%) as pale yellow microcrystals, mp 104° C. (dec).
¹ F. Strain, et al., J. Am. Chem. Soc., 1950, 72, 1254

Di4-nitrobenzyl peroxydicarbonate was found to be a relatively stable material which decomposed as its melting point with slow gas evolution. In comparison, dibenzyl peroxydicarbonate² decomposed with a sudden vigorous expulsion of material from the melting point capillary.
² Cf. M. P. Gore, J. C. Vederas, J. Org. Chem., 1986, 51, 3700

B. 6-(4-Nitrobenzyl peroxydicarbonatyl)-8-[4-[4-(2-pyrimidinyl)piperazinyl]-butyl]-8-azaspiro[4.5]-7,9-dione (II)

To a solution of 8-[4-[4-(2-pyrimidinyl)-piperazinyl]-8-azaspiro[4.5]-7,9-dione (buspirone: 10 g, 26 mmole) in dry THF (250 mL) was added LiN (Me₃Si)₂ (28.5 mL of a 1 M THF solution) at −78° C. and stirred for 3 h and then a solution of di4-nitrobenzyl peroxydicarbonate (11.2 g) in dry THF (150 mL) was added dropwise over 1 h. Stirring was continued at −78° C. for 1 h.

The cooling bath was removed and the reaction solution was poured into a mixture of H₂O and EtOAc. The organic phase was separated and washed with H₂O and then brine. The organic base was dried and then evaporated to a viscous oil. Flash chromatography of this oil, eluting the silica column with MeCN-EtOAc (1:2) gave crude product which was washed with acetone, to remove unreacted buspirone, leaving 6.23 g of a white solid (46%) product (II).

C. 6-Hydroxy-8-[4-[4-(2-pyrimidinyl)-piperazinyl]-butyl]-8-azaspiro[4.5]-7,9-dione (I; 6-HB)

A mixture of 11 (4.0 g; 6.9 mmole) and 10% Pd/C (about 1 g) in MeOH (100 mL) was hydrogenated in a Parr shaker at 40-45 psi for 1 h. The hydrogenation mixture was filtered through a Celite pad which was then washed with EtOAc. The filtrate was evaporated to a gum which was purified by flash chromatography through a silica gel column eluting with EtOAc to give 0.41 g of 6-HB as an off-white solid (I).

Anal. Calcd. for C₂₁H₃₁N₅O₃: C, 62.82; H, 7.78; N, 17.44.

Found: C, 62.84; H, 7.81; N, 17.33.

### Example 2

### One-Pot Synthesis of 6-HB (I)

Buspirone (19.3 g, 50 mmole) was dissolved in dry THF (400 mL) and the resulting solution was cooled to −78° C. A solution of KN(SiMe₃)₂ in toluene (100 mL, 1 M) was added slowly. After the reaction mixture was stirred at −78° C. for 1 h, a solution of 2-(phenylsulfonyl)-3-phenyloxaziridine (Davis reagent, prepared according to literature method: F. A. Davis, et al., Org. Synth., 1988, 66, 203) (17.0 g, 65 mmole) in dry THF (150 mL, precooled to −78° C.) was added quickly via a cannula. After stirring for 30 mins at −78° C., the reaction was quenched with 1 N HCl solution (500 mL). It was extracted with EtOAc (3×500 mL). The aqueous layer was separated, neutralized with saturated sodium bicarbonate solution, and extracted with EtOAc (3×500 mL). The combined organic extracts were dried over Na₂SO₄, filtered, and concentrated under reduced pressure to give a white solid residue which was subjected to column chromatography using CH₂Cl₂/MeOH/NH₄OH (200:10:1) as the eluent to give pure 6-HB (I, 7.2 g) and a mixture of buspirone and 6-HB (I). The mixture was purified by above column chromatography to afford another 3.3 g of pure 6-HB (I).

¹H NMR (CDCl₃) δ 8.30 (d, J=4.7 Hz, 2H), 6.48 (t, J=4.7 Hz, 1H), 4.20 (s, 1H), 3.83-3.72 (m, 5H), 3.55 (s, 1H), 2.80 (d, J=17.5 Hz, 1H), 2.55-2.40 (m, 7H), 2.09-2.03 (m, 1H), 1.76-1.54 (m, 10H), 1.41-1.36 (m, 1H), 1.23-1.20 (m, 1H).

### Example 3

### 5-HT1A Receptor Binding Assay

Membranes are prepared for binding using the human 5-HT1A receptor expressed in HEK293 cells. Cells are collected and ruptured using a dounce homogenizer. The cells are spun at 18000×g for 10 minutes and the pellet is resuspended in assay buffer, frozen in liquid nitrogen and kept at −80° C. until the day of the assay.

A total of 30 ug protein is used per well. The assay is carried out in 96-deep-well plates. The assay buffer is 50 mM HEPES containing 2.5 mM MgCl₂ and 2 mM EGTA. The membrane preparation is incubated at 25° C. for 60 minutes with 0.1 nM to 1000 nM test compound and 1 nM 3H-8-OH-DPAT. 10 mM serotonin serves as blocking agent to determine non-specific binding. The reaction is terminated by the addition of 1 mL of ice cold 50 mM HEPES buffer and rapid filtration through a Brandel Cell Harvester using Whatman GF/B filters. The filter pads are counted in an LKB Trilux liquid scintillation counter. IC₅₀ values are determined using non-linear regression by Excel-fit.

### Example 4

### Proof of Principle Study: Rat Pup Isolation-Induced Ultrasonic Vocalization Test

Harlan Sprague-Dawley rat pups (male and female) were housed in polycarbonate cages with the dam until 9-11 days old. Thirty minutes before testing, pups were removed from the dam, placed into a new cage with a small amount of home bedding and brought into the lab and placed under a light to maintain body temperature at 37° C. Pups were then weighed, sexed, marked and returned to the litter group until behavioral assessment. Testing took place in a Plexiglas recording chamber that contained a metal plate maintained at (18-20° C.) with a 5×5 cm grid drawn on the plate. A microphone was suspended 10 cm above the plate to record ultrasonic vocalizations. Ultrasonic calls were recorded using the Noldus UltraVox system providing on-line analysis of the frequency and duration of calls. The number of grid cells entered by the pup was also collected by visual scoring. Pups that failed to emit at least 60 calls during a 5-minute pretest session were excluded from pharmacological assessment. Immediately following the collection of the baseline measures, pups were injected with vehicle or drug subcutaneously at the nape of the neck and returned to its littermates. Thirty minutes later, pups were retested on each of the dependent measures (vocalization and grid cell crossings) to assess drug effects. Unless otherwise specified, each pup was used only once. Baseline differences and percent change from baseline for the frequency of ultrasonic vocalizations and grid cell crossings were analyzed using a one-way ANOVA. Bonferroni/Dunn post hoc comparisons were performed to assess the acute drug effects with vehicle control. Log-probit analysis was used to estimate the dose (milligrams per kilogram) of each agonist predicted to inhibit isolation-induced ultrasonic vocalizations by 50% (ID₅₀). All comparison were made with an experimental type I error rate (α) set at 0.05.

Doses for each drug were administered in an irregular order across several litters. 6-HB and buspirone were dissolved in physiological saline (0.9% NaCl; vehicle). All injections were administered subcutaneously in a volume of 10 ml/kg. Doses of the drugs refer to the weight of the salt.

1. An improved process for treating a patient suffering from a condition consisting of anxiety, anxiety mixed with depression, depression, and depression mixed with anxiety; the process comprising administration to the patient of an effective amount of pharmaceutically purified 6-hydroxybuspirone or a pharmaceutically acceptable acid addition salt or hydrate thereof.

2. The process of claim 1 for treating a patient suffering from anxiety or anxiety mixed with depression.

3. The process of claim 1 for treating a patient suffering from depression or depression mixed with anxiety.

4. The process of claim 1 wherein the acid addition salt is the hydrochloride.

5. The process of claim 1 wherein the route of administration is selected from the group consisting of oral, sublingual, buccal, transnasal, transdermal or parenteral.

6. The process of claim 5 wherein the route of administration is oral.

7. The use of pharmaceutically purified 6-hydroxybuspirone or a pharmaceutically acceptable salt or hydrate thereof to make a medicament for treating patients suffering from a condition consisting of anxiety, depression, and mixed anxiety and depression.
